(19) United States
(12) Patent Application Publication
(10) Pub. No.: US 2013/0273027 A1
(43) Pub. Date: Oct. 17, 2013

(54) MUTANT PROTEASES AND METHODS OF USE THEREOF

(71) Applicant: Progenra, Inc., Malvern, PA (US)

(72) Inventors: Craig Leach, Ardmore, PA (US); Michael Eddins, West Chester, PA (US); David Stemer, Lansdown, PA (US); Benjamin Nicholson, Merion Station, PA (US)

(21) Appl. No.: 13/799,972

(22) Filed: Mar. 13, 2013

Related U.S. Application Data
(60) Provisional application No. 61/610,186, filed on Mar. 13, 2012.

(51) Int. Cl. C12N 9/48

(52) U.S. Cl. CPC C12N 9/485
USPC 424/94.63; 435/24; 536/23.2; 514/44 R; 435/219

(57) ABSTRACT

Mutant enzymes and methods of use thereof are provided.

This application claims priority under 35 U.S.C. §119(e) to U.S. Provisional Patent Application No. 61/610,186, filed Mar. 13, 2012. The foregoing application is incorporated by reference herein.

### FIELD OF THE INVENTION

The present invention relates to the field of enzymes. More specifically, the instant invention provides mutant proteases which retain enzymatic activity in the absence of cofactors and methods of use thereof.

### BACKGROUND OF THE INVENTION

Several publications and patent documents are cited throughout the specification in order to describe the state of the art to which this invention pertains. Full citations of these references can be found throughout the specification. Each of these citations is incorporated herein by reference as though set forth in full.

A problem with the study of some enzymes, such as certain ubiquitin/ubiquitin-like proteases, is that they are inactive or have poor activity in the absence of cofactors, binding partners, secondary protein modification, or the like. In the case of USP14, the enzyme has little to no activity in the absence of binding to the 26S proteasome. Indeed, upon binding to the 26S proteasome, the activity of USP14 has been reported to be increased by 800-fold (Lee et al. (2010) Nature 467:179-84). The 26S proteasome is a huge complex of approximately 2500 kDa (˜25 MDa) comprising at least 32 different subunits. The purification and use of the 26S proteasome is complicated, cumbersome, and inefficient. Accordingly, improved and more efficient methods for activating USP14 and other ubiquitin proteases are desirable.

### SUMMARY OF THE INVENTION

In accordance with the instant invention, methods for screening for modulators of an enzyme are provided. In a particular embodiment, the method comprises contacting at least one mutant enzyme having at least one blocking loop mutated (e.g., lacking at least one serine of a tetra serine motif in a blocking loop,) with at least one compound (e.g., small molecule); and measuring the activity of the mutant enzyme in the presence of the compound, wherein a modulation in the activity of the mutant enzyme in the presence of the compound compared to the activity of the mutant enzyme in the absence of the compound indicates that the compound is a modulator of the wild-type enzyme. The tetra serine motif may be modified by amino acid insertion, deletion, and/or substitution. In a particular embodiment, the enzyme is an isopeptidase, deubiquinating enzyme, or ubiquitin-like protein (Ubl)-specific proteases (Ulp). In accordance with another aspect of the present invention, nucleic acid molecules encoding an amino acid sequence having at least 80%, 85%, 90%, 95% or more identity with SEQ ID NO: 1 or 2 or a USP14 provided in Table 1 are provided, wherein at least one of the blocking loops has been mutated. In a particular embodiment, at least one serine of the tetra serine motif has been mutated. In a particular embodiment, the nucleic acid molecule is in a vector (e.g., plasmid). Polypeptides comprising a sequence having at least 80%, 85%, 90%, 95% or more identity with SEQ ID NO: 1 or 2 or a USP14 provided in Table 1 are also provided, wherein at least one of the blocking loops has been mutated. In a particular embodiment, at least one serine of the tetra serine motif has been mutated. Kits comprising at least one enzyme mutant (polypeptide or encoding nucleic acid molecule) of the instant invention are also provided.

### BRIEF DESCRIPTION OF THE DRAWINGS

FIG. 1 provides a schematic of the three dimensional structure of USP14 (2AYN). Blocking loop 1 (K334-A341) and blocking loop 2 (G428-G434) are designated. The active site cysteine (C 114) is identified with an asterisk.

FIG. 2 provides sequence alignments of blocking loop 1 and blocking loop 2 of USP14 with known active deubiquitinases (DUBs). Blocking loop 1 sequences are SEQ ID NOs: 10-16 from top to bottom and blocking loop 2 sequences are SEQ ID NOs: 17-23 from top to bottom.

FIG. 3 provides a graph of the activity of USP14 and USP14 mutants. Background (no enzyme) activity is indicated by the dotted line. Data are presented as mean±SD of triplicate determinations.

FIG. 4A provides an amino acid sequence of wild-type USP14 (SEQ ID NO: 1). The underlined amino acids represent the tetra serine motif present in the blocking loop. FIG. 4B provides an amino acid sequence of a mutant USP14 (SEQ ID NO: 2) wherein the tetra serine motif present in the blocking loop has been replaced with the amino acid sequence DHNG (SEQ ID NO: 3).

FIG. 5A provides the amino acid sequence (SEQ ID NO: 24) of N-terminally 6×His- and Smt3-tagged human USP14 protein as it is encoded in pE-SUMOpro-USP14. FIG. 5B provides the nucleotide sequence (SEQ ID NO: 25) of N-terminally 6×His- and Smt3-tagged human USP14 gene as it appears in pE-SUMOpro-USP14. USP14 codons 334-341(blocking loop 1) and 429-433 (blocking loop 2) and the nucleotides that encode them are underlined.

FIGS. 6A and 6B provide DNA sequences of forward (F) and reverse (R) oligonucleotides for USP14 mutagenesis. Flanking sequences are provided in lower case and base changes are in upper case. Sequences are SEQ ID NOs: 26-59 from top to bottom.

FIG. 7 provides a graph of the percentage inhibition of USP14 mutant (BL1-USP21) with PR-619, P22077, IU1 and IU1C. Data normalized relative to DMSO and 10 mM NEM and are expressed as mean±SD of triplicate determinations.

FIG. 8 provides a graph of percentage inhibition of USP14 mutant (BL2-DNHG) with PR-619, P22077, IU1 and IU1C. Data normalized relative to DMSO and 10 mM NEM and are expressed as mean ±SD of triplicate determinations.

FIG. 9 provides a scatterplot of percentage inhibition of USP14 mutant (BL1-USP21) when tested against 50,000 small molecules. Data were normalized relative to DMSO and no enzyme.

FIG. 10 provides a graph of the dose dependent activity of USP14 mutant (BL1-USP21) in the presence of K48-04 IQF diUb. Data are presented as the mean±SD of quadruplicate determinations.

### DETAILED DESCRIPTION OF THE INVENTION

The instant invention provides novel mutated proteases which are active in the absence of a cofactor that is required for the activity of the wild-type protease. As used herein, the term “cofactor” refers to an additional component required for activity of an enzyme. Cofactors may be inorganic or organic. While the term “cofactor” typically refers to a non-protein chemical compound, the term, as used herein, encompasses protein cofactors and binding partners as well. In a particular embodiment, the instant invention provides mutant USP14 molecules which do not require the 26S proteasome as a cofactor for activity. As explained hereinbelow, the mutant proteases of the instant invention may be used, for example, in screening methods for the discovery of modulators of the protease.

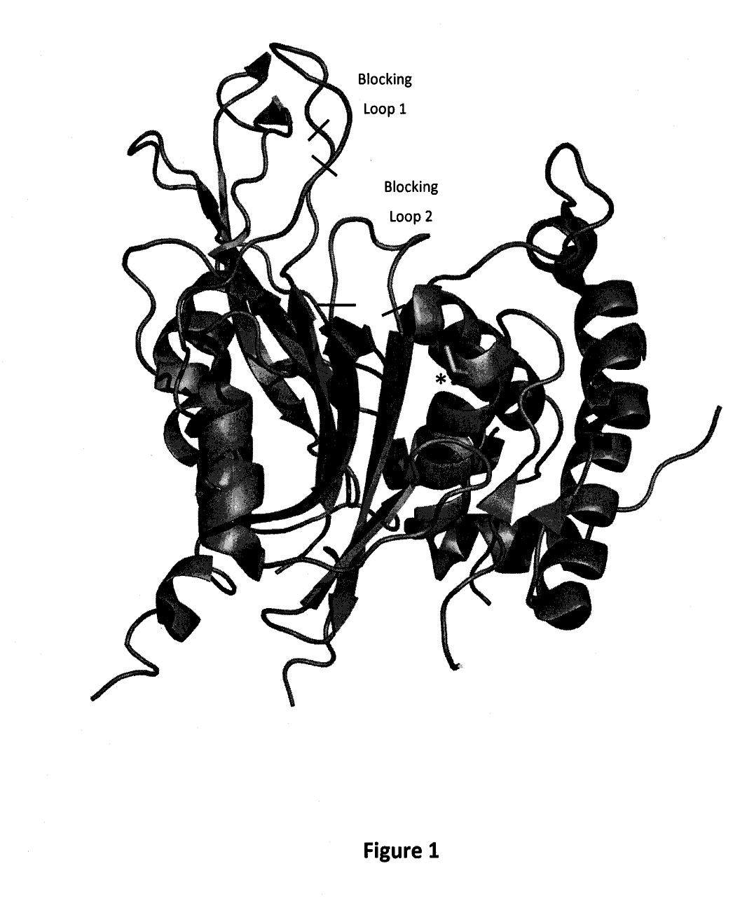

Based on the crystal structure of USP14 (see FIG. 1; Hu et al. (2005) EMBO J., 24:3747-56), it was hypothesized that two peptide loops (blocking loop 1 and blocking loop 2) limit ubiquitin binding to USP14 and that these loops move upon binding to the 26S proteasome. The instant invention demonstrates that the mutation of these “blocking loops” (peptide loops which block/limit/inhibit access to an active site) diminishes their inhibitory effect and increases the catalytic activity of USP14 in the absence of proteasome binding. While USP14 (GenBank Accession No. NP_{—}005142; Gene ID: 9097) is exemplified herein, the instant invention also encompasses mutating blocking loop mutants of other proteases, particularly isopeptidases and deubiquitinating enzymes.

The instant invention encompasses mutant enzymes, particularly proteases, which are active in the absence of a cofactor, binding partner, or secondary protein modification (e.g., post-translational modification) required for activity of the wild-type enzyme. In a particular embodiment, the instant invention encompasses mutant enzymes which are active in the absence of a cofactor (inclusive of protein and non-protein cofactors) which is required for activity of the wild-type enzyme. The wild-type enzyme may have less than 25% activity, less than 10% activity, less than 5% activity, or less than 1% activity in the absence of a cofactor than compared to in the presence of the cofactor. The mutant enzyme of the instant invention may have at least 50%, at least 75%, at least 90%, at least 95%, about 100%, or more than 100% activity in the absence of the cofactor compared to the wild-type enzyme in the presence of the cofactor.

In a particular embodiment, the enzyme of the instant invention is a proteolytic enzyme or isopeptidase. The full-length enzyme may be used or a fragment comprising the catalytically active domain. The enzyme may be from any organism. In a particular embodiment, the enzyme is of human origin. The mutant proteases of the instant invention may comprise at least one affinity tag. In a particular embodiment, the mutant protease is conjugated or linked (e.g., via an amino acid linker (e.g., 1- about 10 amino acids) to SUMO or Smt3. In a particular embodiment, the proteolytic enzyme is a cysteine protease. Cysteine proteases have a catalytic mechanism that involves a cysteine sulfhydryl group. Cysteine proteases include, without limitation, deubiquitinases (DUBs), actinidains, papains, cathepsins, caspases, and calpains. In a particular embodiment, the enzyme of the instant invention is an isopeptidase. Isopeptidases include deubiquitinating enzymes and ubiquitin-like protein (Ubl)-specific proteases (Ulp) (e.g., deSUMOylases, deNEDDylases, delSGylases, and the like). In a particular embodiment, the isopeptidase is a deubiquitinating enzyme. Examples of isopeptidases include, without limitation: ULP1, ULP2, SENP1, SENP2, SENP3, SENP5, SENP6 (aka SUSP1, SSP1), SENP7, NEDD8-specific protease 1 (aka DEN1, Nedp1, Prsc2, SENP8), yeast YUH1, mammalian UCH-L1 (aka Park 5), UCH-L3, UCH-L5 (aka UCH37), USP1 (aka UBP), USP2 (aka UBP41), USP2core, USP2a, USP2b, USP3, USP4 (aka UNP, UNPH), USPS (aka isopeptidase T, ISOT), USP6 (aka TRE2, HRP-1), USP7 (aka HAUSP), USP8 (aka UBPY), USP9, USP9Y (aka DFFRY), USP9X (aka DFFRX), USP10 (aka UBPO, KIAA0190), USP11 (aka UHX1), USP12 (aka USP12L1, UBH1), USP13 (aka ISOT3), USP14 (aka TGT), USP15, USP16 (aka UBP-M), USP18 (aka UBP43, ISG43), USP19 (aka ZMYND9), USP20 (aka VDU1, LSFR3A), USP21, USP22 (aka KIAA1063), USP23, USP24, USP25, USP26, USP27, USP28, USP29, USP30, USP32, USP33 (aka VDU2), USP34, USP35, USP36, USP37, USP38, USP40, USP42, USP44, USP46, USP49, USP51, JosD1 (aka KIAA0063), JosD2 (aka RGD1307305), AMSH, AMSHcore, Ataxin3 (aka ATX3, MJD, MJD1, SCA3, ATXN3), Ataxin3-like, Bap1(UCHL2 or HUCEP-13), DUB-1, DUB-2, DUB1, DUB2, DUB3, DUB4, CYLD, CYLD1, FAFX, FAFY, OTUB1 (aka OTB1, OTU1, HSPC263), OTUB2 (aka OTB2, OTU2, C14orf137), OTU domain containing 7B (aka OTUD7B, Cezanne), KIAA0797, KIAA1707, KIAA0849, KIAA1850, KIAA1850, KIAA0529, KIAA1891, KIAA0055, KIAA1057, KIAA1097, KIAA1372, KIAA1594, KIAA0891, KIAA1453, KIAA1003, UBP1, UBP2, UBP3, UBP4, UBP5, UBP6, UBP7, UBP8, UBP41, UBP43, VCIP135, Tnfaip3 (aka A20), PSMD14 (aka POH1), COP9 complex homolog subunit 5 (aka CSN5, COPS5, JAB1), and YPEL2 (aka FKSG4, and SARS CoV PLpro). Isopeptidases and their nucleic acid coding sequences are well known to those of skill in the art. For use in certain embodiments, isopeptidases can be isolated or recombinantly produced by methods well known in the art.

In a particular embodiment, the proteolytic enzyme/isopeptidase of the instant invention cleaves a ubiquitin or UBL containing substrate. An exemplary amino acid sequence of ubiquitin is the mature human ubiquitin:
MQIFVKTLTG KTITLEVEPS DTIENVKAKI QDKEGIPPDQ QRLIFAGKQL EDGRTLSDYN IQKESTLHLV LRLRGG (SEQ ID NO: 9),
   which is derived by post-translational processing of the naturally occurring human ubiquitin precursor, disclosed at GenBank Accession No CAA44911 (Lund et al., 1985, J. Biol. Chem., 260:7609-7613). In a particular embodiment, the UB or Ubl is the mature form of the protein, i.e., the form of the protein after the precursor has been processed by a hydrolase or peptidase. In particular embodiments, the Ub or Ubl is a mammalian Ub or Ubl, more particularly, a human Ub or Ubl. Ubls include, without limitation, small ubiquitin like-modifier-1 (SUMO), SUMO-2, SUMO-3, SUMO-4, ISG-15, HUB1 (homologous to ubiquitin 1; also known as UBL5 (ubiquitin-like 5)), APG12 (autophagy-defective 12), URM1 (ubiquitin-related modifier 1), NEDD8 (RUB 1), FAT10 (also known as ubiquitin D), and APG8. Amino acid sequences of Ubls and nucleic acid sequences encoding Ubls are known in the art. Amino acid and nucleotide sequences of SUMO proteins are provided, for example, in U.S. Pat. No. 7,060,461 and at GenBank Accession Nos. Q12306 (SMT3; amino acids 1-98 is the mature form), P63165 (SUMO 1; precursor shown, mature form ends in GG), NM_{—}001005781.1 (SUMO1; precursor shown, mature form ends in GG), NP_{—}003343.1 (SUMO1; precursor shown, mature form ends in GG), NM_{—}006937.3 (SUMO2; precursor shown, mature form ends in GG), NM_{—}001005849.1 (SUMO2; precursor shown, mature form ends in GG), NM_{—}006936.2 (SUMO3; precursor shown, mature form ends in GG), and NM_{—}001002255.1 (SUMO4; precursor shown, mature form ends in GG). GenBank Accession No. CAI13493 provides an amino acid sequence for URM1. GenBank Accession No. NP_{—}001041706 provides an amino acid sequence for UBL5 (aka HUB1) (amino acids 1-72 represent the mature form). GenBank GeneID No. 4738 and GenBank Accession No. NP_{—}006147 provide amino acid and nucleotide sequences of NEDD8 (RUB1) (precursor shown, mature form ends in LRGG). GenBank Accession No. P38182 provides an amino acid sequence of yeast ATG8 (aka APG8) (precursor shown, mature form ends in FG). GenBank Accession Nos. BAA36493 and P38316 provide amino acid sequences of human and yeast ATG12 (aka APG12), respectively (human precursor shown, mature form ends in FG). GenBank Accession Nos. AAH09507 and P05161 provide amino acid sequences of human and yeast ISG15 ubiquitin-like modifier, respectively (precursors shown, mature form ends in GG). GenBank Accession No. AAD52982 provides an amino acid sequence of ubiquitin D (aka human FAT10, UBD-3, UBD, GABBR1).

In a particular embodiment, the mutant proteases of the instant invention comprise at least one mutation in at least one blocking loop (i.e., a single mutant may have mutations in one or more blocking loops). The mutation may be an insertion, deletion, and/or substitution mutation. In a particular embodiment, the mutant proteases comprise at least one mutation in a tetra serine motif of the protease, particularly within the blocking loop. In a particular embodiment, the mutant protease comprises at least one, at least two, at least three, or four substitution mutations in the tetra serine motif. The substitution mutations may be conservative or non-conservative. In a particular embodiment, the mutant protease comprises at least one or two acidic amino acids in the tetra serine motif. In a particular embodiment, the mutant protease comprises at least one or two basic amino acids in the tetra serine motif. The mutated tetra serine motif may comprise both acidic and basic amino acids. In a particular embodiment, the tetra serine motif is replaced with the sequence DHNG (SEQ ID NO: 3). Examples of isopeptidases comprising a tetra serine motif in a blocking loop comprise, without limitation (examples of amino acid and nucleotide sequences are provided by GenBank Accession No. and Gene ID No.; location of tetra serine motifs are also provided):

1) ubiquitin specific peptidase 24 (USP24) - GenBank Accession No. NP_{—}056121, Gene ID: 23358, tetra serine motifs at amino acid positions 1050-1053, 1051-1054, 1052-1055, and 1053-1056;

2) USP54—GenBank Accession Nos. NP_{—}689799 and BAH13757, Gene ID: 159195, tetra serine motifs at amino acid positions 566-569, 567-570, 1453-1456, and 1454-1457 of NP_{—}689799 and tetra serine motifs at amino acid positions 566-569 and 567-570 of BAH13757;

3) USP42—GenBank Accession No. EAL23715, Gene ID: 84132, tetra serine motif at amino acid positions 64-67;

4) USP36—GenBank Accession No. NP_{—}079366, Gene ID: 57602, tetra serine motifs at amino acid positions 610-613 and 611-614;
5) USP53—GenBank Accession No. NP_{—}061923; Gene ID: 54532, tetra serine motif at amino acid positions 1000-1003;

6) SUMO1/sentrin specific peptidase 7 (SENP7)—GenBank Accession No. NP_{—}065705, Gene ID: 57337, tetra serine motifs at amino acid positions 202-205;

7) USP26—GenBank Accession No. NP_{—}114113, Gene ID: 83844, tetra serine motif at amino acid positions 185-188;

8) USP10—GenBank Accession No. NP_{—}005144, Gene ID: 9100, tetra serine motif at amino acid positions 352-355;

9) USP51—GenBank Accession No. NP_{—}958443, Gene ID: 158880, tetra serine motif at amino acid positions 93-96;

10) SENP1—GenBank Accession No. NP_{—}055369, Gene ID: 29843, tetra serine motifs at amino acid positions 104-107 and 105-108; and

11) COP9 signalosome complex subunit 5(CSN5)—GenBank Accession No. Q92905, Gene ID: 10987, tetra serine motif at amino acid positions 251-254.

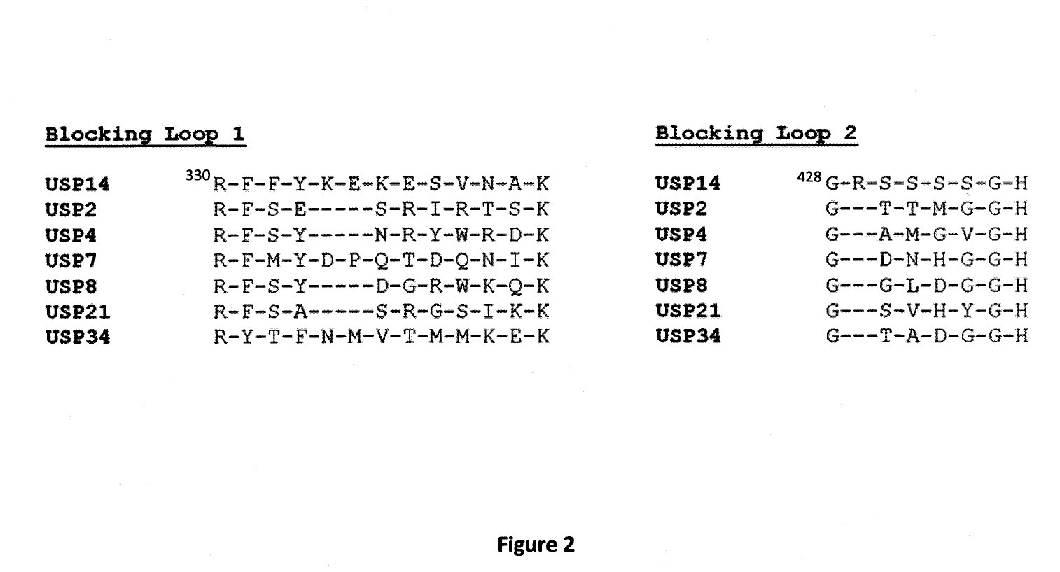

In a particular embodiment, the mutant protease comprises at least one, at least two, at least three, at least four, at least five, at least six, at least seven, or more mutations in a blocking loop. In a particular embodiment, the mutations are substitution mutations. The mutations made within the blocking loops may be such that it changes the amino acid sequence to the corresponding sequence of another protease (see, e.g., FIG. 2). Blocking loops may be identified by sequence alignment with proteases with known blocking loops (see, e.g., FIG. 2). Blocking loops may also be determined by determining the three-dimensional structure of the protease using structural biology means including, without limitation, X-ray diffraction.

In a particular embodiment, the mutant protease of the instant invention is a mutant USP14. In a particular embodiment, the mutant USP14 is a USP14 described in Table 1. The mutant USP14 may have at least 75%, 80%, 85%, 90%, 95%, 97%, 99%, or 100% homology/identity with SEQ ID NO: 2 or a USP14 mutant described in Table 1, particularly at least 90% or 95% homology. In a particular embodiment, at least one amino acid of the tetra serine motif of the USP14 is not a serine (e.g., substituted, deleted, or moved by addition). In a particular embodiment, the tetra serine motif of the mutant USP14 is replaced with the sequence DHNG (SEQ ID NO: 3), TTMG (SEQ ID NO: 4), TADG (SEQ ID NO: 5), or GLDG (SEQ ID NO: 6). In a particular embodiment, at least one amino acid of the first blocking loop of the USP14 is mutated (e.g., by substitution, deletion, or addition). The mutant USP14 may comprise at least one, at least two, at least three, at least four, at least five, at least six, at least seven, or eight mutations in the sequence KEKESVNA (SEQ ID NO: 7) in blocking loop 1. In a particular embodiment, the sequence KEKESVNA (SEQ ID NO: 7) is replaced with the sequence SRGSIK (SEQ ID NO: 8).

As stated hereinabove, mutant proteases may be generated by altering or changing at least one residue in the blocking loop, particularly the tetra serine motif. The residues may be changed to any of the other 20 natural amino acids or to a synthetic or modified amino acid (see, e.g., Table 4 of the MPEP at §2422). The changes may be conservative or non-conservative. A conservative change is the replacement of an amino acid with a one possessing similar properties. For example, Asp and Glu are both acidic amino acids; Lys, Arg, and His are basic amino acids; Asn, Gln, Ser, Thr, and Tyr possess uncharged polar side chains; Ala, Gly, Val, Leu, Ile, Pro, Phe, Met, Trp, and Cys have nonpolar side chains; Ala, Gly, and Leu are small amino acids; Phe, Tyr, and Trp possess large aromatic side chains; and Phe, Tyr, Trp, Val, Ile, and Thr possess bulky uncharged side chains. Accordingly, the replacement of an Asp with a Glu may be considered a conservative change, but replacement of Asp with His would not be a conservative change.

Nucleic acid molecules encoding the mutant enzymes are also encompassed by the instant invention. Nucleic acid molecules encoding the mutant enzymes of the invention may be prepared by any method known in the art. The nucleic acid molecules may be maintained in any convenient vector, particularly an expression vector. Different promoters may be utilized to drive expression of the nucleic acid sequences based on the cell in which it is to be expressed. Antibiotic resistance markers are also included in these vectors to enable selection of transformed cells. Mutant enzyme encoding nucleic acid molecules of the invention include cDNA, DNA, RNA, and fragments thereof which may be single- or double-stranded. The instant invention also encompasses primers, oligonucleotides, probes, antisense molecules, and siRNA molecules directed to or hybridizing with the nucleic acid molecules encoding the mutant enzymes, preferably to the region(s) mutated from the wild-type sequence such that they hybridize preferentially or exclusively to the mutant enzyme compared to the wild-type enzyme.

The present invention also encompasses antibodies capable of immunospecifically binding to a mutant enzyme. Polyclonal and monoclonal antibodies, particularly monoclonal, directed toward a mutant enzyme of the instant invention may be prepared according to standard methods. In a preferred embodiment, the antibodies react immunospecifically with the altered region of the mutant enzyme as compared to the wild-type enzyme. Polyclonal or monoclonal antibodies that immunospecifically interact with mutant enzyme can be utilized for identifying and purifying such proteins. The antibodies may be immunologically specific for the mutant enzyme to the exclusion of wild-type enzyme.

In accordance with another aspect of the instant invention, methods of screening, detecting, and/or identifying modulators of an enzyme are provided. In a particular embodiment, the method comprises contacting at least one mutant enzyme of the instant invention with at least one compound and measuring the activity of the mutant enzyme, wherein a modulation in the activity of the mutant enzyme in the presence of the compound compared to the activity of the mutant enzyme in the absence of the compound indicates that the compound is a modulator of the enzyme. The modulator may be an inhibitor or an enhancer. The method may be a high throughput screening assay.

The compound tested by the methods of the instant invention can be any compound (e.g., an isolated compound), particularly any natural or synthetic chemical compounds (such as small molecule compounds (including combinatorial chemistry libraries of such compounds)), extracts (such as plant-, fungal-, prokaryotic- or animal-based extracts), fermentation broths, organic compounds and molecules, inorganic compound and molecules (e.g., heavy metals, mercury, mercury containing compounds), biological macromolecules (such as saccharides, lipids, peptides, proteins, polypeptides and nucleic acid molecules (e.g., encoding a protein of interest)), inhibitory nucleic acid molecule (e.g., antisense or siRNA), and drugs (e.g., an FDA approved drug). In a particular embodiment, the compound is a small molecule.

The activity of the mutated enzyme may be determined by any means appropriate for the enzyme being investigated. For example, when the enzyme is a proteolytic enzyme, the activity of the enzyme may be determined by contacting the proteolytic enzyme with a substrate of the enzyme and detecting the cleavage of the substrate. The cleavage of the substrate may be measured by direct detection of the cleavage products (e.g., detecting the smaller size fragments of the cleaved substrate (e.g., by SDS-PAGE)). In a particular embodiment, the substrate is operably linked to a detectable label to allow for detection of the cleaved substrate. Detectable labels include, for example, chemiluminescent moieties, bioluminescent moieties, fluorescent moieties, radionuclides, isotopes, radisotopes, and metals. In a particular embodiment, the substrate (e.g., Ub or Ubl) is linked at its C-terminus to an enzyme which requires a free amino-terminus for activity such that the enzyme is detectable only upon cleavage of the substrate (see, e.g., U.S. Pat. No. 7,842,460). In a particular embodiment, the substrate comprises at least one fluorescent moiety (e.g., amino-methylcoumarin (AMC) or rhodamine 110). Such fluorescent moieties allow for the measurement of increased fluorescence intensity as the fluorophore is liberated from the substrate (e.g., Ub/Ubl molecule; see, e.g., Hassiepen et al., 2007, Analyt. Biochem., 371:201-207 and U.S. Pat. No. 4,336,186). The cleavage of the substrate may also be monitored by modulation (loss) of fluorescence resonance energy transfer (FRET). For example, the substrate may be the Ubiquitin LanthaScreen™ reagent available from Invitrogen (Carlsbad, Calif.; U.S. Patent Application Publication No. 2007/0264678). This assay measures fluorescence resonance energy transfer between a fluorophore at the N-terminus of ubiquitin and a second fluorophore at the C-terminus. Also, cleavage of a polyubiquitin substrate can be monitored using the IQF-DiUbiquitin Assay available from Lifesensors, Inc (Malvern, Pa.). In a particular embodiment, luciferase technology may be used to monitor isopeptidase cleavage. In a particular embodiment, the substrate comprises the five C-terminal amino acids of ubiquitin conjugated to an amino-luciferin molecule (DUB-Glo™ (Promega, Inc., Madison, Wis.)). In another embodiment, the substrate comprises the substrate of the enzyme (e.g., Ub or a Ubl) and a luciferase substrate linked to the C-terminus of the Ub or Ubl via an amide linkage (see U.S. patent application Ser. No. 13/157,734). In this assay, the cleavage of the substrate at the C-terminal end of the Ub or Ubl generates free luciferase substrate which can be detecting by luminescence with luciferase, wherein luminescence is indicative of protease activity. The activity of the mutated enzyme can also be measured/detected by measuring the binding of molecules/proteins that bind to the enzyme using biophysical techniques that directly monitor binding of a small molecule/protein to the enzyme such as but not limited to thermal shift assays, isothermal titration calorimetry, surface Plasmon resonance.

Compounds/molecules identified as capable of modulating the activity of particular enzyme using the methods of the present invention can be useful as drugs for the treatment of diseases or conditions associated with a particular enzyme, such as a Ub- or Ubl-specific isopeptidase or its corresponding Ub or Ubl, as well as for further dissecting the mechanisms of action of these enzymes (see, e.g., U.S. patent application Ser. No. 13/168,073).

The present invention also provides compositions comprising at least one mutant enzyme of the instant invention and at least one carrier. The present invention also provides kits for screening, detecting, and/or identifying modulators of an enzyme. In some embodiments, the kits comprise one or more mutant enzymes as described hereinabove (e.g., in one or more composition comprising a carrier). In some embodiments, the kits may further comprise test compounds (e.g., small molecule library) and/or wild-type enzyme. In some embodiments, the kits may further comprise detectable substrates as explained hereinabove. The kits may optionally comprise instructions. Other optional reagents in the kit can include appropriate buffers for enzyme activity. The components of the kits may be contained (individually) in compositions comprising a carrier.

As used herein, “instructions” or “instructional material” includes a publication, a recording, a diagram, or any other medium of expression which can be used to communicate the usefulness of the composition of the invention for performing a method of the invention. The instructions or instructional material of a kit of the invention can, for example, be affixed to a container which contains a kit of the invention to be shipped together with a container which contains the kit. Alternatively, the instructions or instructional material can be shipped separately from the container with the intention that the instructions or instructional material and kit be used cooperatively by the recipient.

### Definitions

The singular forms “a,” “an,” and “the” include plural referents unless the context clearly dictates otherwise.

As used herein, “proteases,” “proteinases” and “peptidases” are interchangeably used to refer to enzymes that catalyze the hydrolysis of covalent peptidic bonds. As used herein, “deubiquitylating enzyme”, “deubiquitinating enzyme” and “DUB” are all used interchangeably to refer to deubiquitinating enzymes.

As used herein, the term “small molecule” refers to a substance or compound that has a relatively low molecular weight (e.g., less than 4,000 Da, particularly less than 2,000 Da). Typically, small molecules are organic, but are not proteins, polypeptides, or nucleic acids, though they may be amino acids or dipeptides.

The term “isolated” may refer to a compound or complex that has been sufficiently separated from other compounds with which it would naturally be associated. “Isolated” is not meant to exclude artificial or synthetic mixtures with other compounds or materials, or the presence of impurities that do not interfere with fundamental activity or ensuing assays, and that may be present, for example, due to incomplete purification, or the addition of stabilizers.

As used herein, a “conservative” amino acid substitution/mutation refers to substituting a particular amino acid with an amino acid having a side chain of similar nature (i.e., replacing one amino acid with another amino acid belonging to the same group). A “non-conservative” amino acid substitution/mutation refers to replacing a particular amino acid with another amino acid having a side chain of different nature (i.e., replacing one amino acid with another amino acid belonging to a different group). Groups of amino acids having a side chain of similar nature are known in the art and include, without limitation, basic amino acids (e.g., lysine, arginine, histidine); acidic amino acids (e.g., aspartic acid, glutamic acid); neutral amino acids (e.g., glycine, asparagine, glutamine, serine, threonine, tyrosine, cysteine, alanine, valine, leucine, isoleucine, proline, phenylalanine, methionine, tryptophan); amino acids having a polar side chain (e.g., asparagine, glutamine, serine, threonine, and tyrosine); amino acids having a non-polar side chain (e.g., alanine, glycine, valine, leucine, isoleucine, proline, phenylalanine, methionine, tryptophan, and cysteine); amino acids having an aromatic side chain (e.g., phenylalanine, tryptophan, histidine); amino acids having a side chain containing a hydroxyl group (e.g., serine, threonine, tyrosine), and the like.

As used herein, “modulate” and “capable of modulating”, in reference to a test agent or agent, includes agents that can increase/enhance or inhibit/decrease/diminish the activity of a particular enzyme. Therefore, screening methods of the present invention are useful for identifying agents that can increase/enhance or inhibit/decrease/diminish the activity of a particular enzyme.

A “carrier” refers to, for example, a buffer, diluent, adjuvant, preservative (e.g., benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent, filler, disintegrant, lubricating agent, binder, stabilizer, or vehicle with which an active agent of the present invention can be contained.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” may refer to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The term “probe” as used herein refers to an oligonucleotide, polynucleotide or DNA molecule, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains about 15 to about 35, about 15 to about 30 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to a DNA oligonucleotide, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically about 10 to about 30 or more, particularly about 15 to about 25, nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The phrases “affinity tag,” “purification tag,” and “epitope tag” may all refer to tags that can be used to effect the purification of a protein of interest. Purification/affinity/epitope tags are well known in the art (see Sambrook et al., 2001, Molecular Cloning, Cold Spring Harbor Laboratory) and include, but are not limited to: polyhistidine tags (e.g. 6×His), polyarginine tags, glutathione-S-transferase (GST), maltose binding protein (MBP), S-tag, influenza virus HA tag, thioredoxin, staphylococcal protein A tag, the FLAG™ epitope, AviTag epitope (for subsequent biotinylation), dihydrofolate reductase (DHFR), an antibody epitope (e.g., a sequence of amino acids recognized and bound by an antibody), the c-myc epitope, and heme binding peptides.

As used herein, the terms “modified,” “engineered,” or “mutant” refer to altered polynucleotide or amino acid sequences. In one embodiment, a polynucleotide sequence encoding an enzyme is modified/engineered/mutated by introducing one or more mutations, particularly by site directed mutagenesis. Additionally, libraries of mutant polynucleotides comprising at least one mutation may also be prepared using random mutagenesis or DNA shuffling techniques. In a particular embodiment, the random mutagenesis is limited to desired regions of the polynucleotide, particularly the region(s) believed to encode the amino acids of the blocking loop and/or tetra serine motif. Common mutagenesis techniques are described in Current Protocols in Molecular Biology, Ausubel, F. et al. eds., John Wiley (2006) and U.S. Pat. Nos. 5,605,793; 5,811,238; 5,830,721; 5,834,252; and 5,837,458. As used herein, a “mutation” or “alteration” refers to a variation in the nucleotide or amino acid sequence of a gene as compared to the naturally occurring or normal nucleotide or amino acid sequence. A mutation may result from the deletion, insertion or substitution of at least one nucleotide or amino acid. In a preferred embodiment, the mutation is a substitution (i.e., the replacement of at least one nucleotide or amino acid with a different nucleotide(s) or amino acid residue(s)).

An “antibody” or “antibody molecule” is any immunoglobulin, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

With respect to antibodies, the term “immunologically specific” refers to antibodies that bind to one or more epitopes of a protein or compound of interest, but which do not substantially recognize and bind other molecules in a sample containing a mixed population of antigenic biological molecules.

The following examples are provided to illustrate various embodiments of the present invention. The examples are illustrative and are not intended to limit the invention in any way.

### EXAMPLE 1

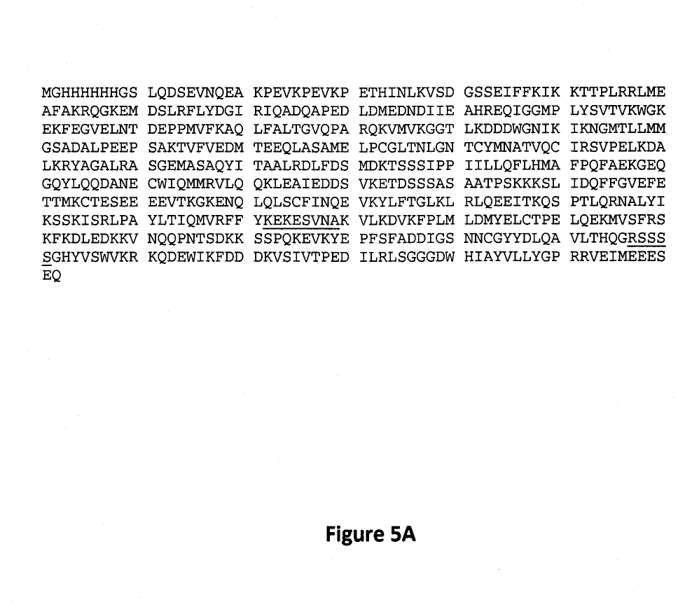

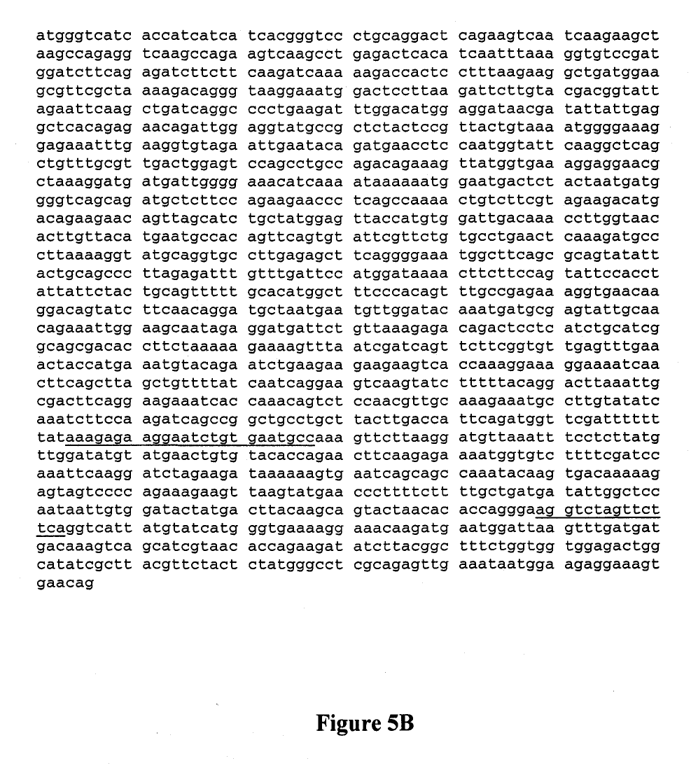

A bacterial expression construct for wild-type human USP14 was prepared by PCR amplification of a full-length cDNA clone encoding full-length USP14 isoform a (GenBank DNA sequence NM_{—}005151.3; GenBank protein sequence NP_{—}005142.1) followed by subcloning into the pE-SUMOpro kan vector (LifeSensors, Inc.; Malvern, Pa.) to generate a plasmid for expression of USP14 with N-terminal 6×His and yeast SUMO (Saccharomyces cerevisiae Smt3) tags. The PCR insert was digested with the restriction enzyme BsaI to generate overhangs complementary to those of BsaI-digested pE-SUMOpro kan, and directional ligation was performed with T4 DNA ligase. After transformation into XL10-Gold® E. coli cells and selection on kanamycin-containing plates, plasmid DNA was prepared, and a correct subclone was verified by DNA sequencing. The resulting plasmid construct was designed for expression of 6×His-Smt3-USP14 via the T7 promoter in E. coli strains containing IPTG-inducible T7 polymerase. Specifically, Rosetta™ 2 (DE3) pLysS cells were used for expression. The predicted sequence of the fusion protein expressed and the DNA sequence encoding it in the plasmid are shown in FIG. 5.

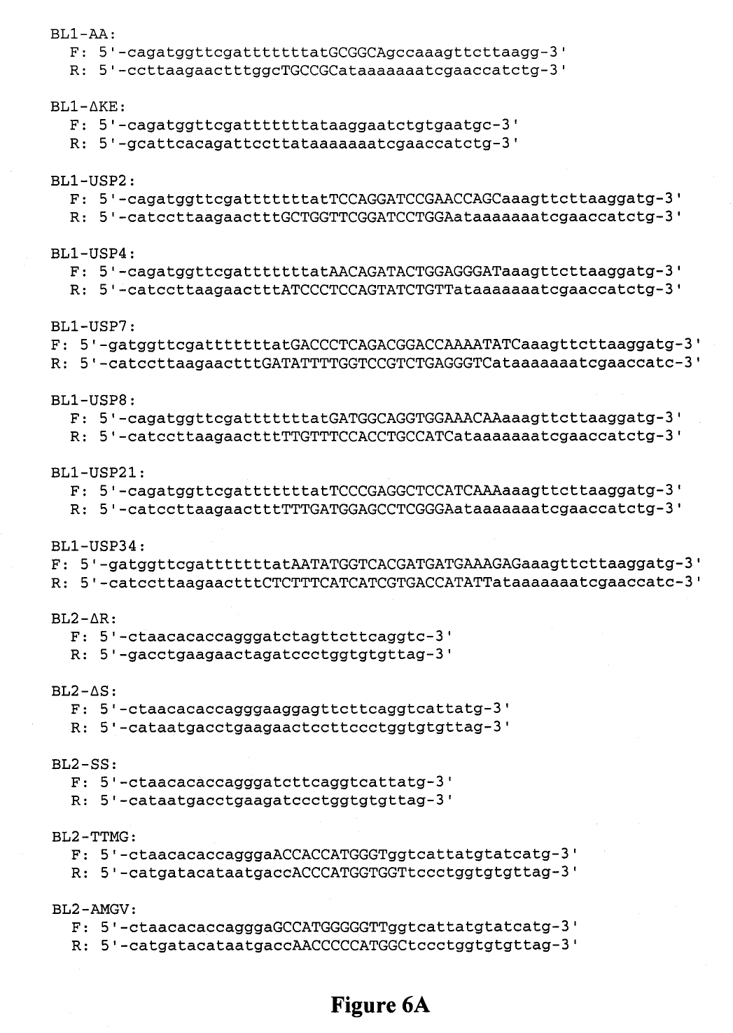

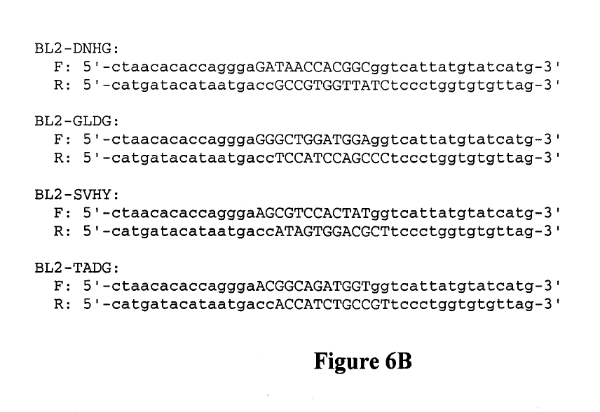

To generate blocking-loop mutants of USP14 in the pE-SUMOpro-USP14 expression construct described above, the QuikChange® (Stratagene/Agilent) method of site-directed mutagenesis was employed. The two areas targeted for mutagenesis were USP14 codons 334-341 (KEKESVNA (SEQ ID NO: 7, an internal portion of blocking loop 1, BL1) and codons 429-433 (RSSSS (SEQ ID NO: 60), blocking loop 2; BL2). These are underlined in FIG. 5. For the QuikChange™ reactions, complementary oligonucleotides were synthesized to contain flanking sequences immediately upstream and downstream of the deletion or base changes to be made in BL1 or BL2. For base changes, the appropriate codons encoding the desired segment of protein sequence were included between the flanking sequences. The list of oligonucleotide sequences is shown in FIG. 6 (flanking sequences are in lower case, and base changes are in upper case). Briefly, pE-SUMOpro-USP14 plasmid was used as a template in reactions containing the complementary QuikChange™ oligos and the DNA polymerase Pfu Turbo® (Stratagene/Agilent), and linear amplification products were obtained by way of appropriate thermocycling. These products were then digested with DpnI restriction enzyme to destroy template plasmid, and the resulting mixtures were transformed into XL10-Gold® E. coli cells and plated on kanamycin-containing media. Small-scale cultures and plasmid preparations were performed on clones from each mutagenesis, and properly mutated plasmids were identified by DNA sequencing. To make the double mutants, the appropriate BL1-mutant plasmid was used as the template for BL2 QuikChange™ mutagenesis.

Expression and purification of protein in E. Coli: An expression construct was generated using SUMOpro fusion vectors (LifeSensors, Inc., Malvern, Pa.) which fused the UBL protein SUMO and a His₆ tag to the N-terminus of USP14 (BL1USP21). Following IPTG induction of mid-log E. coli cultures (Rosetta™) transformed with the expression construct, the 68 kDa His₆-SUMO-USP14 (BL1USP21) fusion protein was detected. Cell pellets were solubilized in buffer containing 50 mM Tris pH 8.0, 500 mM NaCl, 0.25 mM EDTA and sonicated. After centrifugation, His₆-SUMO-USP14 (BL1USP21) fusion protein was purified from the soluble cell lysate by affinity chromatography using Ni-NTA. A HisTrap™ HP (GE Healthcare) column was used equilibrated with start buffer (50 mM Tris pH 8.0, 500 mM NaCl), with washes including 20 mM imidazole and 40 mM imidazole. Elution was carried out in buffer containing 500 mM imidazole. The purified protein was then dialyzed into a storage buffer (20 mM Tris pH 8.0, 150 mM NaCl, 10% glycerol).

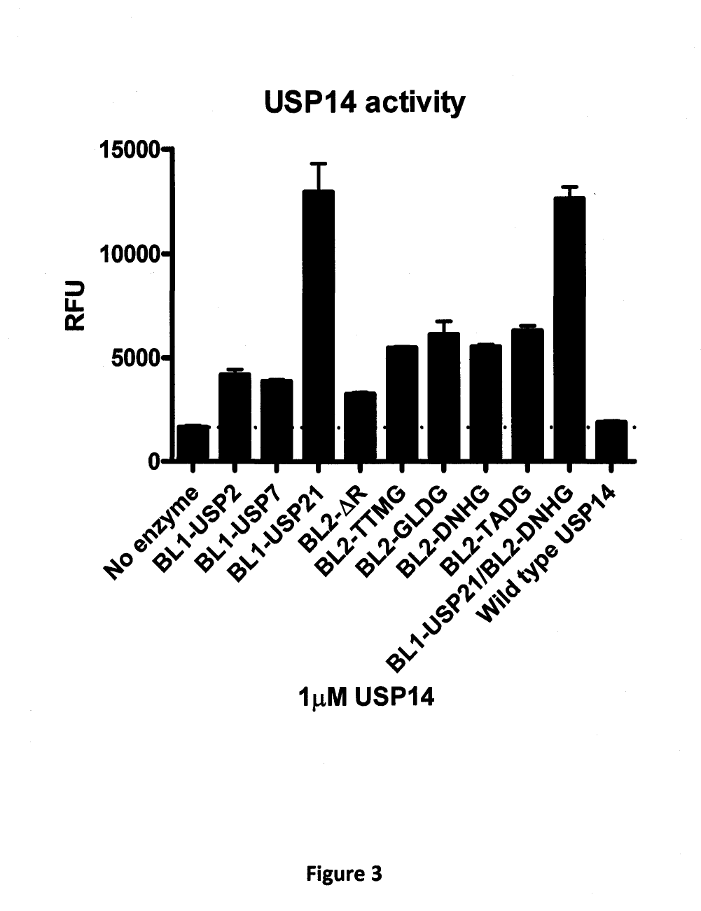

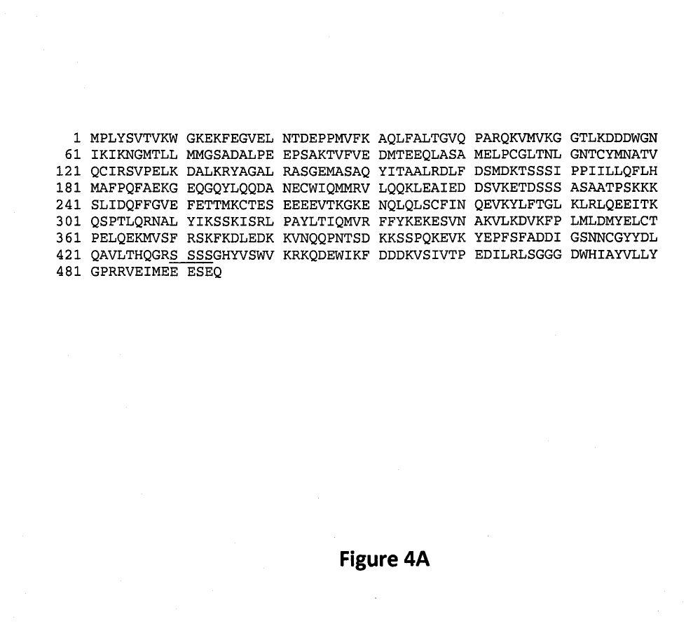

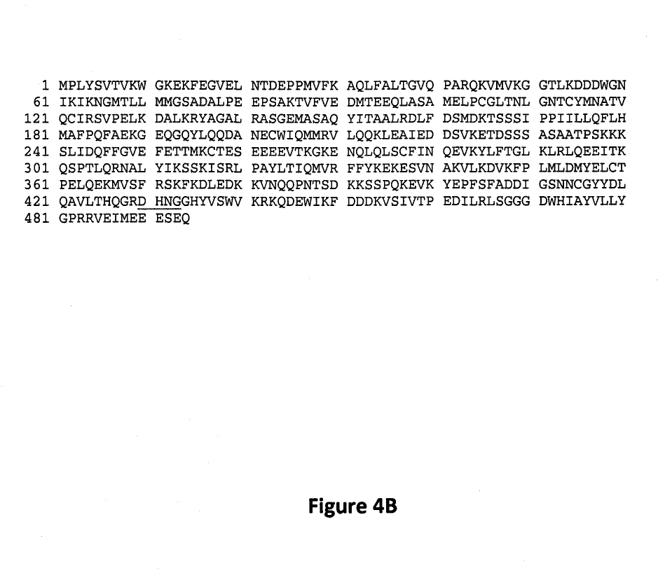

80 μL of buffer, 1.275 μM wild type USP14, or 1.275 μM mutant USP14 were preincubated with 24 DMSO in reaction buffer (20 mM Tris (pH8), 2 mM β-mercaptoethanol and 0.05% CHAPS) for 30 minutes in a black walled 96 well plate. Reactions were initiated by adding 204 of 510 nM ubiquitin-rhodamine 110 (LifeSensors, Cat #S1230; Ub-Rh110) in reaction buffer and incubating the plate at room temperature, protected from ambient light. The final concentrations were 1 μM USP14 enzyme, 1.96% DMSO and 100 nM Ub-Rh110. Reaction progress after two hours was monitored using a fluorimeter equipped with excitation and emission filters of 485 and 528 nm respectively and a 510 nm dichroic mirror. An increase in relative fluorescence units (RFU) relative to buffer alone (no enzyme) was indicative of DUB activity. Table 1 provides the mutations made in USP14. FIG. 3 shows the activity of USP14 and USP14 mutants.

TABLE 1
List of mutations to blocking loops 1 and 2.
The provided sequence for blocking loop 1
begins at amino acid position 334 and the
provided sequence of blocking loop 2
begins at amino acid position 429 of USP14.

| | Blocking | Blocking |
|---|---|---|
| | Loop 1 | Loop 2 |
| Enzyme | (SEQ ID NO) | (SEQ ID NO) |
| wild-type USP14 | KEKESVNA (7) | RSSSS (60) |
| BL1-AA | -----AAA (65) | RSSSS (60) |
| BL1-ΔKE | --KESVNA (66) | RSSSS (60) |
| BL1-USP2 | --SRIRTS (67) | RSSSS (60) |
| BL1-USP4 | --NRYWRD (68) | RSSSS (60) |
| BL1-USP7 | DPQTDQNI (69) | RSSSS (60) |
| BL1-USP8 | --DGRWKQ (70) | RSSSS (60) |
| BL1-USP21 | --SRGSIK (8) | RSSSS (60) |
| BL1-USP34 | NMVTMMKE (71) | RSSSS (60) |
| BL2-ΔR | KEKESVNA (7) | -SSSS (61) |
| BL2-ΔS | KEKESVNA (7) | RSSS- (62) |
| BL2-SS | KEKESVNA (7) | ---SS |
| BL2-TTMG | KEKESVNA (7) | TTMG- (4) |
| BL2-AMGV | KEKESVNA (7) | AMGV- (63) |
| BL2-DNHG | KEKESVNA (7) | DNHG- (3) |
| BL2-GLDG | KEKESVNA (7) | GLDG- (6) |
| BL2-SVHY | KEKESVNA (7) | SVHY- (64) |
| BL2-TADG | KEKESVNA (7) | TADG- (5) |
| BL1-AA/BL2-SS | -----AAA (65) | ---SS |
| BL1-AA/BL2-DNHG | -----AAA (65) | DNHG- (3) |
| BL1-USP21/BL2-DNHG | --SRGSIK (8) | DNHG- (3) |

### EXAMPLE 2

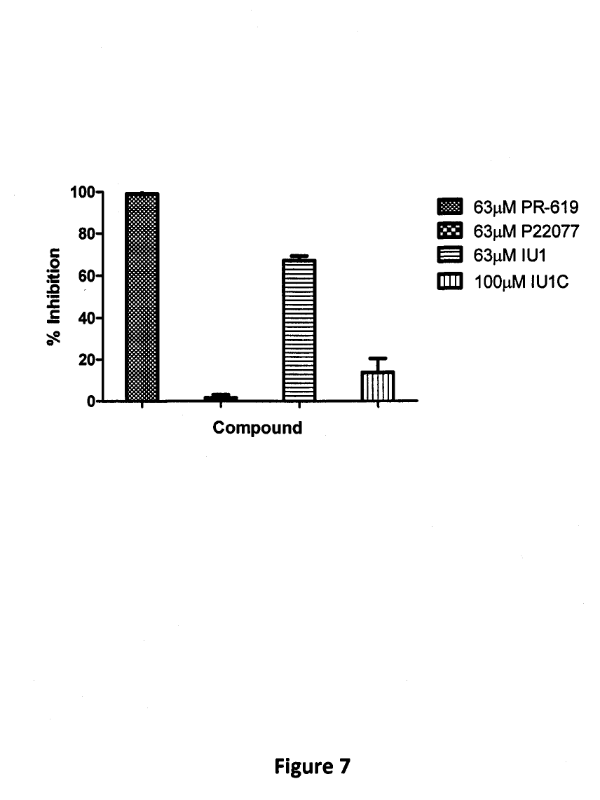

A screening assay was performed with USP14 mutant BL1-USP21. Test compounds were diluted in DMSO to the appropriate concentration before dispensing 2 μl of each compound into a black walled 96 well plate. The compounds were preincubated with 80 μl of reaction buffer containing 63.75 nM mutant USP14 for 30 minutes before adding 20 μl of 510 nM ubiquitin-rhodamine 110 in reaction buffer. The plates were incubated at room temperature before reading on a fluorimeter equipped with excitation and emission filters of 485 and 528 rim respectively and a 510 nm dichroic mirror within the linear range of the assay. Data were normalized relative to the signal from DMSO (0% inhibition) and 10 mM NEM (100% inhibition) containing wells. FIG. 7 provides a graph of the results. As reported, PR-619 and IU1 inhibit USP14 while P22077 and IU do not inhibit USP14 (Altun et al. (2011) Chem. Biol., 18:1401-12; Lee et al. (2010) Nature, 467:179-84).

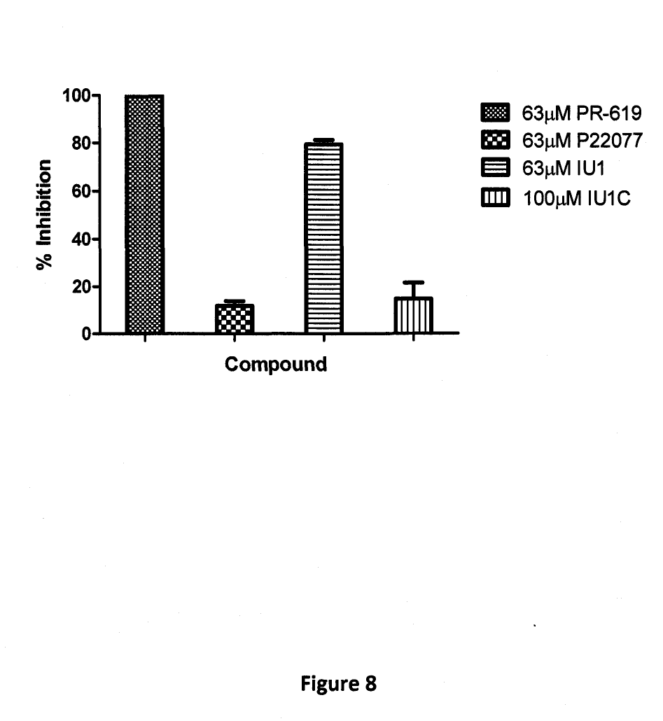

Another screening assay was performed with the USP14 mutant BL2-DNHG. Test compounds were diluted in DMSO to the appropriate concentration before dispensing 2 μl of each compound into a black walled 96 well plate. The compounds were preincubated with 80 μl of reaction buffer containing 1.275 μM mutant USP14 for 30 minutes before adding 20 μl of 510 nM ubiquitin-rhodamine 110 in reaction buffer. The plates were incubated at room temperature before reading on a fluorimeter equipped with excitation and emission filters of 485 and 528 nm respectively and a 510 nm dichroic mirror within the linear range of the assay. These data were normalized relative to the signal from DMSO (0% inhibition) and 10 mM NEM (100% inhibition) containing wells. FIG. 8 provides a graph of the results. As reported, PR-619 and IU1 inhibit USP14 while P22077 and IU do not inhibit USP14 (Altun et al. (2011) Chem. Biol., 18:1401-12; Lee et al. (2010) Nature, 467:179-84).

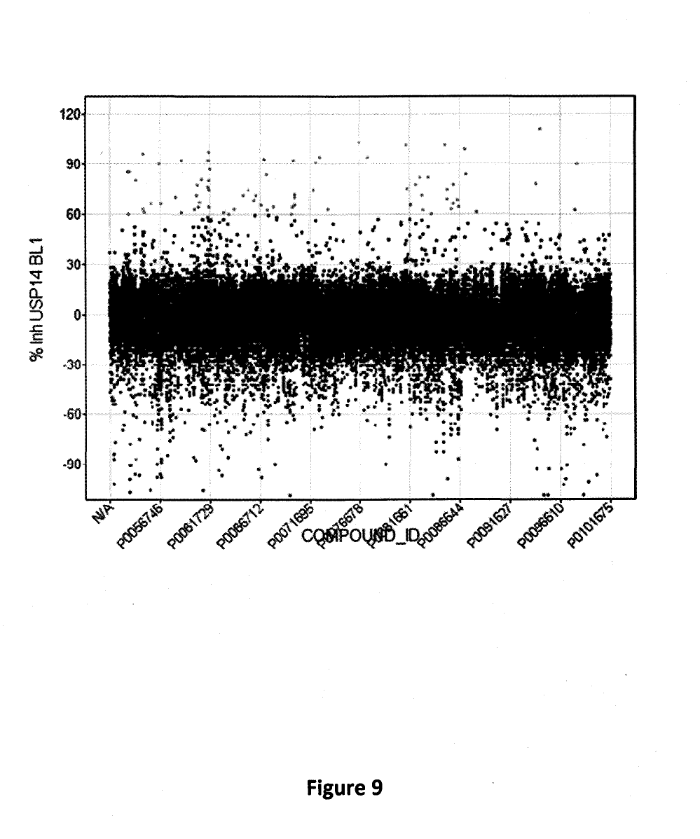

In another screening assay, 50,000 small molecules from LifeChem (Niagara-on-the-Lake, Ontario, Canada) were diluted in DMSO to the appropriate concentration before dispensing into black walled 384 well plates and adding BL1-USP21 mutant USP14 and ubiquitin-rhodamine 110 in reaction buffer. The final concentrations of test compound, BL1-USP21 and ubiquitin-rhodamine 110 were ˜2.5 μM, 80 nM, and 100 nM respectively. The plates were incubated at room temperature before reading on a fluorimeter equipped with excitation and emission filters of 485 and 528 nm respectively and a 510 nm dichroic mirror within the linear range of the assay. These data were normalized relative to the signal from DMSO (0% inhibition) and no USP14 (BL1-USP21) enzyme (100% inhibition) containing wells. As seen in FIG. 9, analysis of these data identified 76 hits that inhibited USP14 (BL1-USP21) activity by >60% (representing ˜3×SD over the median inhibition of the screen).

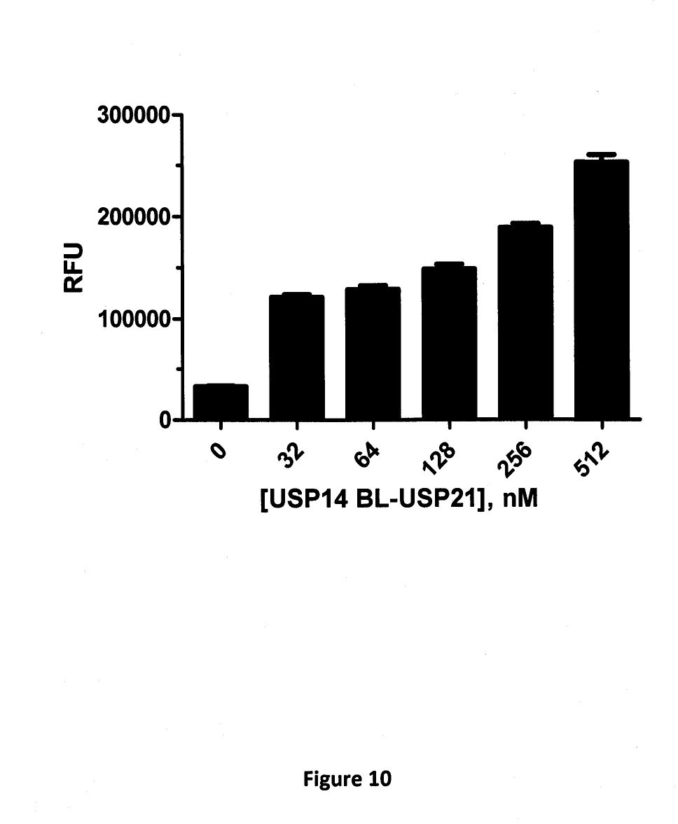

In still another screening assay, dose ranges of BL1-USP21 mutant USP14 were incubated with K48-04 IQF diUb (LifeSensors Inc, Malvern, Pa., USA) in reaction buffer in a black walled 96 well plate. The final concentration of K48-04 IQF diUb was 50 nM. The plate was incubated at room temperature before reading on a fluorimeter equipped with excitation and emission filters of 531 and 590 nm respectively and a general dual dichroic mirror within the linear range of the assay. FIG. 10 illustrates the dose dependent activity of USP14 mutant (BL1-USP21) in the presence of K48-04 IQF diUb.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for screening for modulators of an enzyme, said method comprising
a) contacting at least one mutant of said enzyme with at least one compound, wherein said mutant comprises at least one mutation in at least one blocking loop;
b) measuring the activity of the mutant enzyme in the presence of said compound,
wherein a modulation in the activity of the mutant enzyme in the presence of the compound compared to the activity of the mutant enzyme in the absence of the compound indicates that the compound is a modulator of the wild-type enzyme.

2. The method of claim 1, wherein said modulator is an inhibitor.

3. The method of claim 1, wherein said blocking loop is identified by protein structure.

4. The method of claim 1, wherein said mutation is in a tetra serine motif in said blocking loop.

5. The method of claim 1, wherein said enzyme is an isopeptidase.

6. The method of claim 5, wherein said isopeptidase is a deubiquitinating enzyme or ubiquitin-like protein (Ubl)-specific protease (Ulp).

7. The method of claim 6, wherein said enzyme is selected from the group consisting of ubiquitin specific protease 14 (USP14), USP24, USP42, USP36, USP53, USP26, USP10, USP51, SUMO1/sentrin specific protease 7 (SENP7), SENP1, and COP9 signalsome complex subunit 5 (CSN5).

8. The method of claim 7, wherein said enzyme is USP14.

9. The method of claim 8, wherein said mutant enzyme comprises an amino acid sequence having at least 80% homology with SEQ ID NO: 1 or 2, wherein at least one amino acid of the tetra serine motif is not a serine.

10. The method of claim 9, wherein said mutant enzyme comprises SEQ ID NO: 2.

11. The method of claim 8, wherein said wherein said mutant enzyme comprises an amino acid sequence having at least 80% homology with SEQ ID NO: 1 or 2, wherein at least one amino acid of the sequence KEKESVNA (SEQ ID NO: 7) is changed.

12. The method of claim 1, wherein said compound is a small molecule.

13. An isolated nucleic acid molecule encoding an amino acid sequence having at least 80% homology with SEQ ID NO: 2 or a USP14 provided in Table 1.

14. The isolated nucleic acid molecule of claim 13, wherein at least one amino acid of the tetra serine motif is not a serine.

15. The isolated nucleic acid molecule of claim 13, wherein at least one amino acid of the sequence KEKESVNA (SEQ ID NO: 7) is changed.

16. A composition comprising an isolated nucleic acid molecule of claim 13 and a pharmaceutically acceptable carrier.

17. An isolated polypeptide comprising a sequence having at least 80% homology with SEQ ID NO 2 or a USP14 provided in Table 1.

18. The isolated polypeptide of claim 17, wherein at least one amino acid of the tetra serine motif is not a serine.

19. The isolated polypeptide of claim 17, wherein at least one amino acid of the sequence KEKESVNA (SEQ ID NO: 7) is changed.

20. A composition comprising an isolated polypeptide of claim 17 and a pharmaceutically acceptable carrier.

21. A kit comprising at least one isolated polypeptide of claim 17 and, optionally, at least one detectable substrate.

22. The kit of claim 21, wherein said detectable substrate is a fluorescently labeled ubiquitin.


SEQUENCE LISTING (71 sequences)

SEQ ID NO: 1 - protein, 494 aa, Homo sapiens
MPLYSVTVKWGKEKFEGVELNTDEPPMVFKAQLFALTGVQPARQKVMVKGGTLKDDDWGNIKIKNGMTLLMMGSADALPEEPSAKTVFVEDMTEEQLASAMELPCGLTNLGNTCYMNATVQCIRSVPELKDALKRYAGALRASGEMASAQYITAALRDLFDSMDKTSSSIPPIILLQFLHMAFPQFAEKGEQGQYLQQDANECWIQMMRVLQQKLEAIEDDSVKETDSSSASAATPSKKKSLIDQFFGVEFETTMKCTESEEEEVTKGKENQLQLSCFINQEVKYLFTGLKLRLQEEITKQSPTLQRNALYIKSSKISRLPAYLTIQMVRFFYKEKESVNAKVLKDVKFPLMLDMYELCTPELQEKMVSFRSKFKDLEDKKVNQQPNTSDKKSSPQKEVKYEPFSFADDIGSNNCGYYDLQAVLTHQGRSSSSGHYVSWVKRKQDEWIKFDDDKVSIVTPEDILRLSGGGDWHIAYVLLYGPRRVEIMEEESEQ

SEQ ID NO: 2 - protein, 494 aa, Artificial Sequence
  mutant USP14
MPLYSVTVKWGKEKFEGVELNTDEPPMVFKAQLFALTGVQPARQKVMVKGGTLKDDDWGNIKIKNGMTLLMMGSADALPEEPSAKTVFVEDMTEEQLASAMELPCGLTNLGNTCYMNATVQCIRSVPELKDALKRYAGALRASGEMASAQYITAALRDLFDSMDKTSSSIPPIILLQFLHMAFPQFAEKGEQGQYLQQDANECWIQMMRVLQQKLEAIEDDSVKETDSSSASAATPSKKKSLIDQFFGVEFETTMKCTESEEEEVTKGKENQLQLSCFINQEVKYLFTGLKLRLQEEITKQSPTLQRNALYIKSSKISRLPAYLTIQMVRFFYKEKESVNAKVLKDVKFPLMLDMYELCTPELQEKMVSFRSKFKDLEDKKVNQQPNTSDKKSSPQKEVKYEPFSFADDIGSNNCGYYDLQAVLTHQGRDHNGGHYVSWVKRKQDEWIKFDDDKVSIVTPEDILRLSGGGDWHIAYVLLYGPRRVEIMEEESEQ

SEQ ID NO: 3 - protein, 4 aa, Artificial Sequence
  mutant tetra serine motif
DHNG

SEQ ID NO: 4 - protein, 4 aa, Artificial Sequence
  mutant tetra serine motif
TTMG

SEQ ID NO: 5 - protein, 4 aa, Artificial Sequence
  mutant tetra serine motif
TADG

SEQ ID NO: 6 - protein, 4 aa, Artificial Sequence
  mutant tetra serine motif
GLDG

SEQ ID NO: 7 - protein, 8 aa, Artificial Sequence
  blocking loop 1 motif
KEKESVNA

SEQ ID NO: 8 - protein, 6 aa, Artificial Sequence
  mutant blocking loop 1 motif
SRGSIK

SEQ ID NO: 9 - protein, 76 aa, Homo sapiens [sequence omitted]

SEQ ID NO: 10 - protein, 13 aa, Artificial Sequence
  USP14 blocking loop 1 motif
RFFYKEKESVNAK

SEQ ID NO: 11 - protein, 11 aa, Artificial Sequence
  USP2 blocking loop 1 motif
RFSESRIRTSK

SEQ ID NO: 12 - protein, 11 aa, Artificial Sequence
  USP4 blocking loop 1 motif
RFSYNRYWRDK

SEQ ID NO: 13 - protein, 13 aa, Artificial Sequence
  USP7 blocking loop 1 motif
RFMYDPQTDQNIK

SEQ ID NO: 14 - protein, 11 aa, Artificial Sequence
  USP8 blocking loop 1 motif
RFSYDGRWKQK

SEQ ID NO: 15 - protein, 11 aa, Artificial Sequence
  USP21 blocking loop 1 motif
RFSASRGSIKK

SEQ ID NO: 16 - protein, 13 aa, Artificial Sequence
  USP34 blocking loop 1 motif
RYTFNMVTMMKEK

SEQ ID NO: 17 - protein, 8 aa, Artificial Sequence
  USP14 blocking loop 2 motif
GRSSSSGH

SEQ ID NO: 18 - protein, 7 aa, Artificial Sequence
  USP2 blocking loop 2 motif
GTTMGGH

SEQ ID NO: 19 - protein, 7 aa, Artificial Sequence
  USP4 blocking loop 2 motif
GAMGVGH

SEQ ID NO: 20 - protein, 7 aa, Artificial Sequence
  USP7 blocking loop 2 motif
GDNHGGH

SEQ ID NO: 21 - protein, 7 aa, Artificial Sequence
  USP8 blocking loop 2 motif
GGLDGGH

SEQ ID NO: 22 - protein, 7 aa, Artificial Sequence
  USP21 blocking loop 2 motif
GSVHYGH

SEQ ID NO: 23 - protein, 7 aa, Artificial Sequence
  USP34 blocking loop 2 motif
GTADGGH

SEQ ID NO: 24 - protein, 602 aa, Artificial Sequence
  6xHis- and Smt3-tagged human USP14 protein
MGHHHHHHGSLQDSEVNQEAKPEVKPEVKPETHINLKVSDGSSEIFFKIKKTTPLRRLMEAFAKRQGKEMDSLRFLYDGIRIQADQAPEDLDMEDNDIIEAHREQIGGMPLYSVTVKWGKEKFEGVELNTDEPPMVFKAQLFALTGVQPARQKVMVKGGTLKDDDWGNIKIKNGMTLLMMGSADALPEEPSAKTVFVEDMTEEQLASAMELPCGLTNLGNTCYMNATVQCIRSVPELKDALKRYAGALRASGEMASAQYITAALRDLFDSMDKTSSSIPPIILLQFLHMAFPQFAEKGEQGQYLQQDANECWIQMMRVLQQKLEAIEDDSVKETDSSSASAATPSKKKSLIDQFFGVEFETTMKCTESEEEEVTKGKENQLQLSCFINQEVKYLFTGLKLRLQEEITKQSPTLQRNALYIKSSKISRLPAYLTIQMVRFFYKEKESVNAKVLKDVKFPLMLDMYELCTPELQEKMVSFRSKFKDLEDKKVNQQPNTSDKKSSPQKEVKYEPFSFADDIGSNNCGYYDLQAVLTHQGRSSSSGHYVSWVKRKQDEWIKFDDDKVSIVTPEDILRLSGGGDWHIAYVLLYGPRRVEIMEEESEQ

SEQ ID NO: 25 - DNA, 1806 bp, Artificial Sequence
  6xHis- and Smt3-tagged human USP14
atgggtcatcaccatcatcatcacgggtccctgcaggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcaggcccctgaagatttggacatggaggataacgatattattgaggctcacagagaacagattggaggtatgccgctctactccgttactgtaaaatggggaaaggagaaatttgaaggtgtagaattgaatacagatgaacctccaatggtattcaaggctcagctgtttgcgttgactggagtccagcctgccagacagaaagttatggtgaaaggaggaacgctaaaggatgatgattggggaaacatcaaaataaaaaatggaatgactctactaatgatggggtcagcagatgctcttccagaagaaccctcagccaaaactgtcttcgtagaagacatgacagaagaacagttagcatctgctatggagttaccatgtggattgacaaaccttggtaacacttgttacatgaatgccacagttcagtgtattcgttctgtgcctgaactcaaagatgcccttaaaaggtatgcaggtgccttgagagcttcaggggaaatggcttcagcgcagtatattactgcagcccttagagatttgtttgattccatggataaaacttcttccagtattccacctattattctactgcagtttttgcacatggctttcccacagtttgccgagaaaggtgaacaaggacagtatcttcaacaggatgctaatgaatgttggatacaaatgatgcgagtattgcaacagaaattggaagcaatagaggatgattctgttaaagagacagactcctcatctgcatcggcagcgacaccttctaaaaagaaaagtttaatcgatcagttcttcggtgttgagtttgaaactaccatgaaatgtacagaatctgaagaagaagaagtcaccaaaggaaaggaaaatcaacttcagcttagctgttttatcaatcaggaagtcaagtatctttttacaggacttaaattgcgacttcaggaagaaatcaccaaacagtctccaacgttgcaaagaaatgccttgtatatcaaatcttccaagatcagccggctgcctgcttacttgaccattcagatggttcgatttttttataaagagaaggaatctgtgaatgccaaagttcttaaggatgttaaatttcctcttatgttggatatgtatgaactgtgtacaccagaacttcaagagaaaatggtgtcttttcgatccaaattcaaggatctagaagataaaaaagtgaatcagcagccaaatacaagtgacaaaaagagtagtccccagaaagaagttaagtatgaacccttttcttttgctgatgatattggctccaataattgtggatactatgacttacaagcagtactaacacaccagggaaggtctagttcttcaggtcattatgtatcatgggtgaaaaggaaacaagatgaatggattaagtttgatgatgacaaagtcagcatcgtaacaccagaagatatcttacggctttctggtggtggagactggcatatcgcttacgttctactctatgggcctcgcagagttgaaataatggaagaggaaagtgaacag

SEQ ID NO: 26 - DNA, 43 bp, Artificial Sequence
  BL1-AA forward primer
cagatggttcgatttttttatgcggcagccaaagttcttaagg

SEQ ID NO: 27 - DNA, 43 bp, Artificial Sequence
  BL1-AA reverse primer
ccttaagaactttggctgccgcataaaaaaatcgaaccatctg

SEQ ID NO: 28 - DNA, 38 bp, Artificial Sequence
  BL1-.delta.KE forward primer
cagatggttcgatttttttataaggaatctgtgaatgc

SEQ ID NO: 29 - DNA, 38 bp, Artificial Sequence
  BL1-.delta.KE reverse primer
gcattcacagattccttataaaaaaatcgaaccatctg

SEQ ID NO: 30 - DNA, 55 bp, Artificial Sequence
  BL1-USP2 forward primer
cagatggttcgatttttttattccaggatccgaaccagcaaagttcttaaggatg

SEQ ID NO: 31 - DNA, 55 bp, Artificial Sequence
  BL1-USP2 reverse primer
catccttaagaactttgctggttcggatcctggaataaaaaaatcgaaccatctg

SEQ ID NO: 32 - DNA, 55 bp, Artificial Sequence
  BL1-USP4 forward primer
cagatggttcgatttttttataacagatactggagggataaagttcttaaggatg

SEQ ID NO: 33 - DNA, 55 bp, Artificial Sequence
  BL1-USP4 reverse primer
catccttaagaactttatccctccagtatctgttataaaaaaatcgaaccatctg

SEQ ID NO: 34 - DNA, 59 bp, Artificial Sequence
  BL1-USP7 forward primer
gatggttcgatttttttatgaccctcagacggaccaaaatatcaaagttcttaaggatg

SEQ ID NO: 35 - DNA, 59 bp, Artificial Sequence
  BL1-USP7 reverse primer
catccttaagaactttgatattttggtccgtctgagggtcataaaaaaatcgaaccatc

SEQ ID NO: 36 - DNA, 55 bp, Artificial Sequence
  BL1-USP8 forward primer
cagatggttcgatttttttatgatggcaggtggaaacaaaaagttcttaaggatg

SEQ ID NO: 37 - DNA, 55 bp, Artificial Sequence
  BL1-USP8 reverse primer
catccttaagaactttttgtttccacctgccatcataaaaaaatcgaaccatctg

SEQ ID NO: 38 - DNA, 55 bp, Artificial Sequence
  BL1-USP21 forward primer
cagatggttcgatttttttattcccgaggctccatcaaaaaagttcttaaggatg

SEQ ID NO: 39 - DNA, 55 bp, Artificial Sequence
  BL1-USP21 reverse primer
catccttaagaacttttttgatggagcctcgggaataaaaaaatcgaaccatctg

SEQ ID NO: 40 - DNA, 59 bp, Artificial Sequence
  BL1-USP34 forward primer
gatggttcgatttttttataatatggtcacgatgatgaaagagaaagttcttaaggatg

SEQ ID NO: 41 - DNA, 59 bp, Artificial Sequence
  BL1-USP34 reverse primer
catccttaagaactttctctttcatcatcgtgaccatattataaaaaaatcgaaccatc

SEQ ID NO: 42 - DNA, 31 bp, Artificial Sequence
  BL2-.delta.R forward primer
ctaacacaccagggatctagttcttcaggtc

SEQ ID NO: 43 - DNA, 31 bp, Artificial Sequence
  BL2-.delta.R reverse primer
gacctgaagaactagatccctggtgtgttag

SEQ ID NO: 44 - DNA, 37 bp, Artificial Sequence
  BL2-.delta.S forward primer
ctaacacaccagggaaggagttcttcaggtcattatg

SEQ ID NO: 45 - DNA, 37 bp, Artificial Sequence
  BL2-.delta.S reverse primer
cataatgacctgaagaactccttccctggtgtgttag

SEQ ID NO: 46 - DNA, 31 bp, Artificial Sequence
  BL2-SS forward primer
ctaacacaccagggatcttcaggtcattatg

SEQ ID NO: 47 - DNA, 31 bp, Artificial Sequence
  BL2-SS reverse primer
cataatgacctgaagatccctggtgtgttag

SEQ ID NO: 48 - DNA, 44 bp, Artificial Sequence
  BL2-TTMG forward primer
ctaacacaccagggaaccaccatgggtggtcattatgtatcatg

SEQ ID NO: 49 - DNA, 44 bp, Artificial Sequence
  BL2-TTMG reverse primer
catgatacataatgaccacccatggtggttccctggtgtgttag

SEQ ID NO: 50 - DNA, 44 bp, Artificial Sequence
  BL2-AMGV forward primer
ctaacacaccagggagccatgggggttggtcattatgtatcatg

SEQ ID NO: 51 - DNA, 44 bp, Artificial Sequence
  BL2-AMGV reverse primer
catgatacataatgaccaacccccatggctccctggtgtgttag

SEQ ID NO: 52 - DNA, 44 bp, Artificial Sequence
  BL2-DNHG forward primer
ctaacacaccagggagataaccacggcggtcattatgtatcatg

SEQ ID NO: 53 - DNA, 44 bp, Artificial Sequence
  BL2-DNHG reverse primer
catgatacataatgaccgccgtggttatctccctggtgtgttag

SEQ ID NO: 54 - DNA, 44 bp, Artificial Sequence
  BL2-GLDG forward primer
ctaacacaccagggagggctggatggaggtcattatgtatcatg

SEQ ID NO: 55 - DNA, 44 bp, Artificial Sequence
  BL2-GLDG reverse primer
catgatacataatgacctccatccagccctccctggtgtgttag

SEQ ID NO: 56 - DNA, 44 bp, Artificial Sequence
  BL2-SVHY forward primer
ctaacacaccagggaagcgtccactatggtcattatgtatcatg

SEQ ID NO: 57 - DNA, 44 bp, Artificial Sequence
  BL2-SVHY reverse primer
catgatacataatgaccatagtggacgcttccctggtgtgttag

SEQ ID NO: 58 - DNA, 44 bp, Artificial Sequence
  BL2-TADG forward primer
ctaacacaccagggaacggcagatggtggtcattatgtatcatg

SEQ ID NO: 59 - DNA, 44 bp, Artificial Sequence
  BL2-TADG reverse primer
catgatacataatgaccaccatctgccgttccctggtgtgttag

SEQ ID NO: 60 - protein, 5 aa, Artificial Sequence
  USP14 blocking loop 2 motif
RSSSS

SEQ ID NO: 61 - protein, 4 aa, Artificial Sequence
  BL2-.delta.R blocking loop 2 motif
SSSS

SEQ ID NO: 62 - protein, 4 aa, Artificial Sequence
  BL2-.delta.S blocking loop 2 motif
RSSS

SEQ ID NO: 63 - protein, 4 aa, Artificial Sequence
  BL2-AMGV blocking loop 2 motif
AMGV

SEQ ID NO: 64 - protein, 4 aa, Artificial Sequence
  BL2-SVHY blocking loop 2 motif
SVHY

SEQ ID NO: 65 - protein, 3 aa, Artificial Sequence
  blocking loop 1 motif
AAA

SEQ ID NO: 66 - protein, 6 aa, Artificial Sequence
  BL1-.delta.KE blocking loop 1 motif
KESVNA

SEQ ID NO: 67 - protein, 6 aa, Artificial Sequence
  BL1-USP2 blocking loop 1 motif
SRIRTS

SEQ ID NO: 68 - protein, 6 aa, Artificial Sequence
  BL1-USP4 blocking loop 1 motif
NRYWRD

SEQ ID NO: 69 - protein, 8 aa, Artificial Sequence
  BL1-USP7 blocking loop 1 motif
DPQTDQNI

SEQ ID NO: 70 - protein, 6 aa, Artificial Sequence
  BL1-USP8 blocking loop 1 motif
DGRWKQ

SEQ ID NO: 71 - protein, 8 aa, Artificial Sequence
  BL1-USP34 blocking loop 1 motif
NMVTMMKE